(19) United States
(12) Patent Application Publication
(10) Pub. No.: US 2005/0165446 A1
(43) Pub. Date: Jul. 28, 2005

(54) Personal piercing hygiene aid

(76) Inventor: Kelly B. Craft, Odem, TX (US)

Correspondence Address: Natan Wesely, Suite 1100, 1001 McKinney, Houston, TX, 77002, US

(21) Appl. No.: 10/765,557

(22) Filed: Jan. 27, 2004

(51) Int. Cl. A61B 17/04

(52) U.S. Cl. 606/228

(57) ABSTRACT

A piercing hygiene system to cleanse and/or treat body piercings. The hygiene system comprises a curved blunt insert attached to spongy elongated material that can be used to clean and treat body piercings. The spongy material is saturated with an antimicrobial, a topical anesthetic, a flavored saline gel, or an essential oil such as rosemary, lemongrass, tea tree oil, rosewood oil, or thyme oil. Additionally, the blunt insert is curved so the insert may penetrate piercings that are flat against the body, such as eyebrows, or are difficult to access, such as belly button piercings, or are through thick tissue, such as tongue piercings.

### FIELD OF INVENTION

The invention relates to a cleaning system for body piercing. More specifically, the invention relates to a curved blunt plastic insert to which is attached spongy elongated material that can be used to clean and treat body piercings.

### BACKGROUND OF THE INVENTION

Cleaning systems for body piercings are necessary to clean and prevent infection. It is known in the prior art to provide a mechanism to clean the inside of a piercing, such as pierced ears.

Some examples of such cleaning systems known in the prior art are as follows. U.S. Pat. No. 4,798,216 to McCarty et al. teaches a thread impregnated with hypo-allergenic, anti-bacterial astringent for cleansing and conditioning pierced ear lobes. The thread has no mechanism for insertion into and through piercings. These aspects are undesirable to the user.

U.S. Pat. No. 4,497,402 to Karos teaches an apparatus for cleaning and sterilizing ear lobe holes for pierced earrings that utilizes a string of absorbent material having attached to a firm tip. The firm tip is of limited use in piercings other ear lobes because of the limited space, shape of the piercing, or amount of tissue to penetrate in piercings such as eyebrow and tongue piercings. These aspects are undesirable to the user.

U.S. Pat. No. 6,146,398 to Satterfield teaches a hand manipulated tool to cleanse and apply medication to a pierced body hole. The tool has two sections, the first section is a cleansing section, and the second section has a medication absorbent layer. This tool is of limited use in piercings other than ear lobes because of the limited space, shape of the piercing, or amount of tissue to penetrate in piercings such as eyebrow and tongue piercings. These aspects are undesirable to the user.

It is desirable to have a mechanism to clean and treat piercings, such as tongue piercings, that require a curved mechanism to navigate the piercing. It also is desirable to have a mechanism to clean, debride, and treat piercings that curve through the body part, such as eyebrow piercings.

### SUMMARY OF THE INVENTION

The present invention is directed primarily toward a mechanism to clean and treat body piercings, but may be adapted for other medical uses. The preferred embodiment of the present invention is a local delivery system for body piercing consisting of a curved blunt insert attached to spongy flexible elongated material. The spongy material can be treated with an antimicrobial, a topical anesthetic, a flavored saline gel, or an essential oil such as rosemary, lemongrass, tea tree oil, rosewood oil, or thyme oil to deliver such treatment to the adjacent tissue. Additionally, the blunt insert is curved so the insert may penetrate piercings that are flat against the body, such as eyebrows, or are difficult to access, such as belly button piercings, or are through thick tissue, such as tongue piercings.

An object of the present invention is to provide an original and effective cleansing and treatment delivery system for body piercings.

### BRIEF DESCRIPTION OF THE DRAWINGS

FIG. 1 is a plan view of the delivery system for body piercing.

### DETAILED DESCRIPTION OF THE INVENTION

Detailed embodiments of the present invention are disclosed herein. It is to be understood, however, that the disclosed embodiments are merely exemplary of the invention that may be embodied in various and alternative forms. The figures are not necessarily to scale where some features may be exaggerated or minimized to show details of particular components. Therefore, specified structural and functional details disclosed herein are not to be interpreted as limiting, but merely as a basis for the claims and as a representative basis for teaching one skilled in the art to variously employ the present invention.

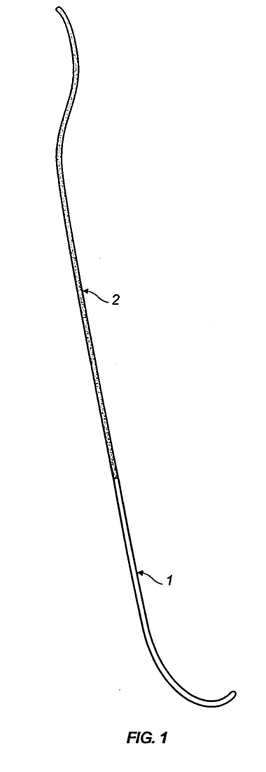

Referring to FIG. 1, the curved blunt insert 1 can be formed from smooth flexible, semi-flexible, formable or rigid materials such as plastic, surgical steel, wire encased plastic, carbon fiber and the like. The curved blunt insert 1 may range in size from gauge 2 to gauge 20. The curved blunt insert 1 is tapered enough to penetrate a variety of piercings, but not pointed enough to break the skin of a user when applied with moderate force. In a preferred embodiment, the curved blunt insert 1 is formed from plastic material as a single piece. In a preferred embodiment, the curved blunt insert 1 has sufficient curvature to enter, travel and exit a piercing located in an eye brow, tongue, belly button, or other soft tissue and/or cartilage without injuring adjacent tissue or creating a deviant piercing off of the piercing.

The spongy flexible elongated material 2 is attached to the curved blunt insert 1. FIG. 1 depicts a preferred embodiment wherein the spongy flexible elongated material 2 is a length of yarn. The spongy flexible elongated material 2 made from a flexible material of a pre-determined length. The spongy flexible elongated material 2 is of sufficient cross section to pass through a piercing to cleanse but not damage adjacent tissue. The spongy flexible elongated material 2 can be yarn, gauze, suture material, nylon fiber and foam, or a combination thereof. In a preferred embodiment, spongy flexible elongated material 2 is capable of holding and thereafter delivering by contact at least one treatment to adjacent tissue. In such an embodiment, spongy flexible elongated material 2 is capable of absorbing a treatment and thereafter delivering it to adjacent tissue as it passes through a piercing. In a preferred embodiment, spongy flexible elongated material 2 is a spongy flexible elongated material saturated with at least one treatment selected from a group consisting of antimicrobial, topical anesthetic, flavored saline gel, and/or essential oils—such as rosemary, lemongrass tea tree oil, rosewood oil or thyme oil.

The curved blunt insert 1 is inserted through a body piercing, pulling the spongy flexible elongated material 2 through the body piercing, thereby cleaning and debriding the body piercing and treating the interior of the body piercing with the a variety of products, including an antimicrobial, a topical anesthetic, a flavored saline gel, or an essential oil such as rosemary, lemongrass, tea tree oil, rosewood oil, or thyme oil.

With respect to the above description, it is to be realized that the optimum dimensional relationships for the parts of the invention, to include variations in size, materials, shape, form, function and manner of operations, assembly and use, are deemed readily apparent and obvious to one skilled in the art, and all equivalent relationships to those illustrated in the drawings, and described in the specification, are intended to be encompassed by the present invention. Therefore, the foregoing is considered as illustrative only of the principles of the invention. Further, since numerous modifications and changes will readily occur to those skilled in the art, it is not desired to limit the invention to the exact construction and operation shown and described. Accordingly, all suitable modifications and equivalents are considered to fall within the scope of the invention.

1. A piercing hygiene system comprising:
a curved blunt insert with two ends, said first end is pointed and said second end is fixed to a spongy elongated material wherein said insert can be inserted through a body piercing pulling the spongy elongated material through a body piercing to clean, debride, and treat the body piercing.

2. A piercing hygiene system as claimed in claim 1, wherein said swab is further configured to deliver at least one treatment selected from a group consisting of antimicrobial, topical anesthetic, flavored saline gel, and essential oil to adjacent tissue.

3. A body piercing cleaning and treatment system as claimed in claim 1, wherein said spongy elongated material is saturated with a substance.

4. A body piercing cleaning and treatment system as claimed in claim 3, wherein said substance is selected from the group consisting of an antimicrobial, a topical anesthetic, a flavored saline gel, and an essential oil such as rosemary, lemongrass, tea tree oil, rosewood oil, and thyme oil.
